(19) United States
(12) Patent Application Publication
(10) Pub. No.: US 2024/0235064 A9
(43) Pub. Date: Jul. 11, 2024

(54) Method for Producing a Local Coil for an MRT Measurement

(71) Applicant: Siemens Healthcare GmbH, Erlangen (DE)

(72) Inventor: Stephan Biber, Erlangen (DE)

(73) Assignee: Siemens Healthcare GmbH, Erlangen (DE)

(21) Appl. No.: 18/381,718

(22) Filed: Oct. 19, 2023

(30) Foreign Application Priority Data
Oct. 21, 2022 (DE) 10 2022 211 217.2

(51) Int. Cl. H01Q 21/28; H01Q 21/00

(52) U.S. Cl. CPC H01Q 21/28; H01Q 21/0087

(57) ABSTRACT

A method for producing a local coil for an MRT measurement, including shaping a coil structure from an elongate conductor; shaping an antenna for the MRT measurement from the coil structure; introducing the antenna into a cavity mold that is designed to receive a liquid plastic material, wherein the antenna is arranged in the cavity mold in such a way that it is at least partially encapsulatable by liquid plastic material; shaping a local coil by filling the cavity mold with a liquid plastic material and curing the plastic material.

### TECHNICAL FIELD

The disclosure relates to a method for producing a local coil for an MRT measurement (MRT: Magnetic Resonance Tomography) and to a local coil produced in such a way. The disclosure particularly relates to a monolithic production method for single-use MR local coils (MR: Magnetic Resonance).

### BACKGROUND

Imaging teeth and the human jaw is practiced only to a limited extent on modern-day MR systems. This is partly because alternative imaging techniques are usually employed (X-ray), MR is not readily available in a dental medicine environment, and the patients are generally not referred to radiology.

However, MR methods offer significant advantages in dental technology that lie not only in the dose-free imaging but also in the possibility of visualizing cysts or detecting degradation of dentin before this becomes recognizable through X-rays, e.g., using ultrashort TE (or zero TE) imaging.

Optimal image quality, in particular with respect to the signal-to-noise ratio, is obtained when a special coil is used for dental imaging. Such special coils should be used in the lower magnetic field strength range (0.2 T-0.7 T) since less tissue magnetization by the basic field is present. With such coils, however, strict attention must be given to hygiene since these are situated in a patient's oral cavity during a scan and can be contaminated there with or pass on the bacteria. This constitutes a serious disadvantage for reusable coils.

Similar problems arise for coils intended for MR examination of other body orifices or for coils inserted into blood vessels.

Typically, oral coils consist of an antenna, electronic modules (for detuning and possibly preamplifiers), connecting elements such as cables or plugs, and a housing. These four components are assembled, with the functional components installed in the housing and connected.

### SUMMARY

It is an object of the present aspects of the disclosure to disclose a method for producing a local coil for an MRT measurement, which overcomes the shortcomings of the prior art and permits the simple and cost-effective production of a local coil. This enables single-use coils to be provided to maintain the highest hygienic standards.

This object is achieved using a method according to claim 1 and a local coil according to claim 11.

The method according to the aspects of the disclosure for producing a local coil for an MRT measurement, comprises the following steps:
shaping a coil structure from an elongate conductor,
shaping an antenna for the MRT measurement from the coil structure, e.g., by bending the coil structure,
introducing the antenna into a cavity mold that is designed to receive a liquid plastic material, the antenna being arranged in the cavity mold in such a way that it can be at least partially encapsulated by liquid plastic material,
shaping a local coil by filling the cavity mold with a liquid plastic material and curing the plastic material.

The shaping of the coil structure is known per se in the prior art. For this purpose, a conductor made of metal (in particular, a highly conductive metal) is wound to form a coil, which typically has a number of turns. More information on the general shape and the windings of a suitable coil may be found in, e.g., the publication DE 10 2011 079 577 A1.

The coil structure may comprise a single coil or a plurality of coils. For example, the coil structure may be implemented as a 1-, 2- or 4-channel coil, wherein a single-channel coil should cover the entire jaw (top and bottom), in a two-channel coil, each channel should cover a part of the jaw, e.g., top/bottom or right/left, and in a 4-channel coil each of the coils should cover a quarter of the jaw, e.g., top left, top right, bottom left and bottom right. In the case of multichannel coils, the use of parallel imaging techniques (SENSE, GRAPPA, or derivatives) is possible. With multichannel coils, a decoupling is preferred to be realized inductively by overlapping the coils. In the case of 2-channel coils, the overlap should lie in the mouth region, and with 4-channel coils in the region of the mouth and the occlusal plane.

In the case of an oral coil, the coil structure should follow the shape of the jaw. To that end, the coil structure is bent to a desired curvature. It is by all means possible, in this case, for different coil structures to have different radii of curvature so that they are suitable for different jaws. After all, patients should be able to wear the coils in comfort. For example, a greater curvature (and, if necessary, smaller coils) can be used for children than adults. If the coil structure lies on a plane, the radius of curvature naturally lies perpendicularly or at least obliquely to said plane.

However, the antenna can also be shaped using other treatments, e.g., by compressing into an elongate shape, coating, or contacting terminals. Theoretically, the coil structure per se may constitute the antenna, though this rarely occurs in practice.

The reworked coil structure, e.g., bent and/or provided with terminals, is now referred to as an “antenna” and, in theory, can be used as such for an MRT measurement. According to the aspects of the disclosure, however, said antenna is now additionally encased with a plastic material, which serves to protect the antenna and allows the local coil to be worn comfortably. The antenna enveloped in this way is then referred to as a “local coil.”

For the enveloping process, the antenna is inserted into a cavity mold designed to receive a liquid plastic material, i.e., it can be filled with a liquid plastic material. To ensure the antenna is subsequently at least partially surrounded by the plastic material, it is introduced into the cavity mold so that it can be at least partially enveloped by liquid plastic material. This can be achieved, e.g., by the antenna being secured at two points on the periphery of the cavity mold, specifically at its two terminals, and otherwise being disposed freely in the interior of the cavity mold. The antenna can be fixed in place in the cavity mold or can be joined to the mold in a positive-fitting manner at certain points using its own shape.

The local coil is then shaped by filling the cavity mold with a liquid plastic material and curing the plastic material. This process is known per se, e.g., from injection molding. However, the plastic material does not necessarily have to be heated. It would also be possible, e.g., to use a two-component resin that, after mixing, is poured into the cavity mold and cures there.

Using this method, a special intraoral dental local coil is obtained, which can be realized easily and cost-effectively. As a result, it is possible, for example, to produce these local coils as disposable coils. Because they are shaped as dental coils, in particular on account of the curvature, a higher image quality can be achieved than with extraoral coils. It is preferred in this case that the curvature of the antenna is such that teeth and jaw are covered. Although the antenna can cover the temporomandibular joint or other parts of the head or deglutition apparatus, covering the teeth is preferred.

A local coil, according to the aspects of the disclosure, has been shaped using a method according to the aspects of the disclosure.

Further particularly advantageous embodiments and developments of the aspects of the disclosure will become apparent from the dependent claims as well as from the following description, wherein the independent claims of one claims category may also be developed analogously to the dependent claims and exemplary embodiments of a different claims category, and in particular individual features of different exemplary embodiments or variants may also be combined to form new, exemplary embodiments or variants.

According to a preferred method, the latter is combined with a design mold to shape the antenna. Said design mold can have hard or elastic walls and, by all means, be or include the cavity mold. The said combination preferably comprises that the antenna be plastically deformed by the design mold by compression molding, in particular with the application of heat. It is preferred in this case that the design mold be removed again following said deformation of the antenna, particularly in the case in which the coil structure is self-supporting. The design mold, therefore, serves here to shape the antenna following the molding of the coil structure. However, it is not absolutely necessary for the design mold to be used only for shaping the antenna. If it constitutes a cavity mold, it can, by all means, shape the antenna (e.g., by bending the coil structure) and, after that, be used as a mold for the plastic material.

However, the combination can also preferably comprise that the antenna be joined to the design mold in such a way, preferably by clamping, compressing, or adhesive bonding, that the antenna is held in shape using the design mold during the curing of the plastic material. This alternative is particularly advantageous when the antenna is not self-supporting. The design mold can then preferably be encased with plastic material, and the antenna in a cavity mold.

According to a preferred method, the conductor is a wire, a flexible lead, or a conductor track on a substrate material. Alternatively, the coil structure is formed from a continuous conductor. It is preferred in this case that the coil structure comprises a plurality of conductors that form a plurality of independent and electrically isolated antenna elements.

According to a preferred method, the local coil is shaped using an injection molding process, a casting process, a foaming process, or a dip molding process. In this case, the cured plastic material preferably has a modulus of elasticity of less than 3 GPa and/or comprises a compressible foam material. Preferably, at least the plastic material serving to shape the outer surface of the local coil is biocompatible (the local coil is intended to be introduced into a body orifice for a measurement).

According to a preferred method, the antenna is shaped in such a way and is introduced into the cavity mold so that end pieces of the conductor protrude from the cavity mold and are not encased by plastic material during the shaping of the local coil. The local coil can now be connected to an electronics module (an “interface”) with said exposed metal surfaces serving as contacts.

According to a preferred method, the antenna is shaped in such a way and is introduced into the cavity mold so that the antenna forms an inductance that can be inductively connected to an external inductance.

According to a preferred method, the antenna is shaped in such a way and is introduced into the cavity mold so that the antenna forms an electrode that can be used according to its intended purpose for capacitive coupling to an external electrode. It is preferred in this case that the plastic material or a substrate material of a conductor track of the conductor serves as a dielectric, preferably wherein the conductor track with the substrate material protrudes from the plastic housing (i.e., the cured plastic material) such that only the dielectric surface of the substrate material appears externally.

Preferred means of coupling the local coil to an MR system or an interface are conductive coupling, inductive coupling, and capacitive coupling. Regardless of the type of coupling, it is preferred that the local coil is connected to the active electronics (externally) via a plug in the mouth region. An inductive or capacitive coupling, in particular via electrodes or external coil elements, can be advantageous for hygiene reasons and on workflow/comfort grounds.

According to a preferred method, an insertion element is additionally introduced into the cavity mold before it is filled with the plastic material. Alternatively or, in addition, the cavity mold is cast in such a way that (after being filled with the liquid plastic material) a cavity is created inside the resulting local coil. This is advantageous to reduce the weight of the local coil or provide the mold with a higher degree of elasticity. In this case, said hollow local coil is basically produced in one manufacturing step and has no external seam, which is advantageous in terms of hygiene. An actively acting element, in particular an electronic component such as, e.g. an RFID chip, could also be introduced into such a cavity.

According to a preferred method, the step of filling the mold with a liquid plastic material is repeated a number of times such that different plastic layers are superimposed on one another. In this case, it is preferred that the cavity mold is swapped between two filling steps. Preferably, a plurality of different plastic materials possessing different elastic properties in the cured state are used in this case. Using such a multistage manufacturing process and as a result of using more and less elastic materials, advantageous mechanical properties of the resulting local coil can be produced, e.g., a compressibility to allow better contouring to shape. It is also possible in this way to create cavities and to embed additional elements, such as, e.g. RFID transponders, into the local coil. This embodiment variant is particularly advantageous for non-self-supporting coils. Firstly, the coil is bent using the cavity mold to form the antenna and embedded into a first matrix (as a design mold) made of a first plastic material. A second matrix made of an identical or different plastic material, which is shaped in a second cavity mold, then provides for a desired surface of the local coil. It can be advantageous if an outermost encasement is formed using a plastic material with particular biocompatibility.

According to a preferred method, a plurality of local coils of the same type are manufactured in different sizes. In this case, it is preferred that the local coils be produced so that they have terminals at different geometric positions. An interface (for securing the local coil) can then be configured so that it has a plurality of possible terminals. It should be noted that coils with different sizes often also have different impedances. Thus, if coils of identical size or identical impedance have identical terminals and coils of different size or having different impedance have different terminals, the interface can be designed so that it performs a suitable impedance transformation according to the terminal used to provide the same impedance at all times to the downstream electronics regardless of which size variant is currently connected. This variant is very easy to implement and to handle during application.

According to a preferred method, the antenna is produced in such a way that it forms an additional inductance, particularly at a point with little imaging impact. Such a point of little imaging impact is located in particular at a part of the antenna that protrudes out of the mouth after insertion of the antenna into the oral cavity. The additional inductance is preferably a coil that has its surface normal in the z-direction, i.e., along the radius of curvature of the curvature of the coil body to the antenna.

The normal on the coil plane is preferably oriented in the x direction (laterally) or in the y-direction (in the region of the incisors), whereby the coil with its main field picks up the RF fields of the spins oriented in the transverse direction (x-y) and consequently possesses very high sensitivity (as desired) in this region. It is also preferred that antennas or the elements thereof are arranged tilted slightly out of the x-z plane to permit better contouring to shape (to fit the palate). A further illumination in the direction of the roots could also be achieved therewith. The coordinate system is based on the usual MRT coordinates.

In the manufacture of a plurality of local coils of the same type, though the antennas thereof are of different sizes, the antennas of said local coils are preferably produced having different additional inductances. In this case, the differences in the additional inductances are such that the smallest antenna receives the greatest additional inductance. For example, the local coil can be provided in different sizes for different anatomies (XL, L, M, S, XS). In this case, all the outer dimensions, preferably scale, but not all by the same factor.

According to a preferred method, the local coil is manufactured to comprise a shortening capacitor, which is designed and connected to the antenna so that the antenna has a predetermined impedance. A shortening capacitor is a capacitor that serves for electrically shortening the antenna. It is preferably connected in series with the antenna. It is of advantage if the capacitor is of the highest possible quality. To avoid electric charges, it is preferred to connect a surge protection device to the circuit in parallel with the shortening capacitor. It is particularly preferred for the shortening capacitor to be integrated into the antenna conductor (i.e., the antenna that is manufactured from the conductor), in particular using a double-sided metallization. Alternatively, or in addition, it is preferred that the shortening capacitor is produced using a discrete element.

A preferred local coil comprises contacts for electrical contacting with an MRT interface and mechanical retaining structures which, in conjunction with the contacts, are designed to form a plug that serves to produce a mechanically reinforced plug-in connection with the interface. Plugs on local coils are known per se in the prior art. In this case, however, the cavity mold is preferably formed so that a plug is produced as early as during the shaping of the local coil by filling the cavity mold with a liquid plastic material.

A preferred local coil is embodied with regard to its antenna and the shape of the enveloping plastic material for insertion into a body orifice, particularly for insertion into the oral cavity or for rectal or vaginal insertion, or insertion into a blood vessel.

A preferred local coil comprises mechanical or electrical structures at its surface, which are formed in such a way that they can serve as triggers for a switch of an MRT interface or as electrical signal contacts. This has, e.g., the advantage that the successful insertion of the coil can be signaled to an interface, e.g., in the form of a leading pin for the mechanical or electrical triggering of a switching operation in the interface.

With an oral coil, it is preferred that the antenna or the coil elements are arranged in the cheek pouches, e.g., between teeth/jaw and inside of the cheek. Alternatively, the antenna can also be arranged internally in the mouth between the dental arches, e.g., between the tongue and teeth/jaw.

The local coil is preferably implemented as an RX or RX/TX coil.

The antenna is preferably designed to be provided in particular for field strengths in the range of 0.15-0.8T. Due to the low base SNR, the proximity of the coil to the region of interest is of particular significance there.

Preferably, the local coil contains no active electronics. This enables very cost-effective production.

### BRIEF DESCRIPTION OF THE DRAWINGS

The disclosure's aspects are explained again below in more detail with the aid of exemplary embodiments and with reference to the attached figures like components in the different figures are labeled with identical reference numerals. The figures are generally not to scale. In the figures:

FIG. 1 shows a schematic representation of a magnetic resonance tomography system according to an exemplary embodiment of the aspects of the disclosure,

FIG. 2 shows a local coil comprising two antennas and a bite splint,

FIG. 3 shows an arrangement of the local coil in an oral cavity from above,

FIG. 4 shows a preferred antenna mold for a local coil for use in the oral cavity,

FIG. 5 shows a preferred double antenna mold for a local coil for use in the oral cavity,

FIG. 6 shows a preferred quadruple antenna mold for a local coil for use in the oral cavity and

FIG. 7 shows a block diagram of an exemplary embodiment of a method according to the aspects of the disclosure.

Only elements essential to the aspects of the disclosure or helpful for an understanding thereof are depicted in the figures.

### DETAILED DESCRIPTION

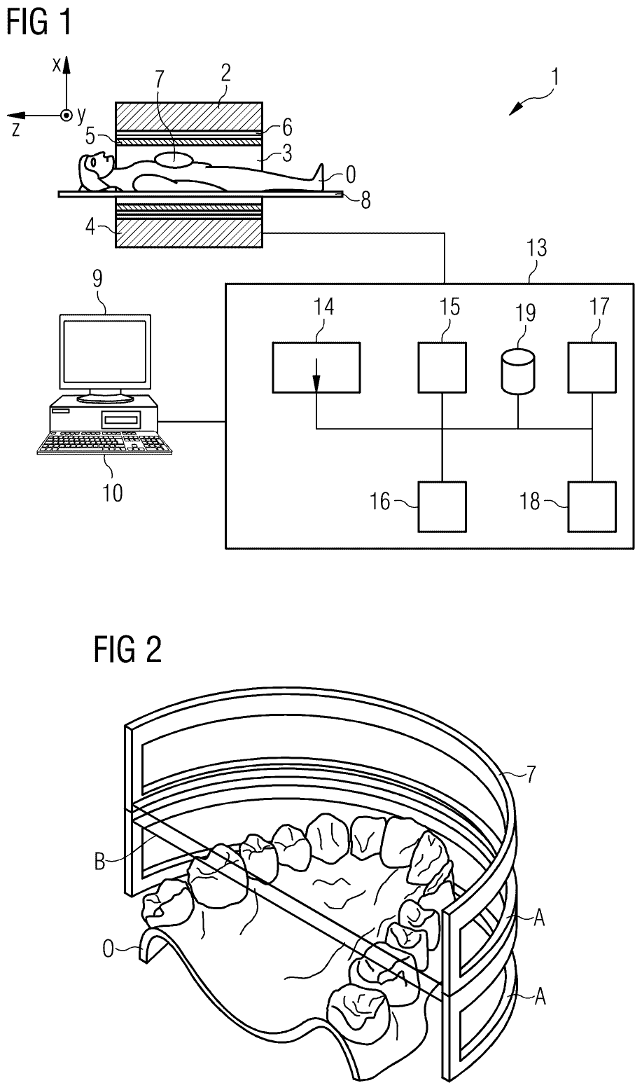

FIG. 1 shows a roughly schematic view of a magnetic resonance tomography system 1 (MRT system). It comprises the actual magnetic resonance scanner 2 primarily with an examination chamber 3 or patient tunnel into which a patient or test volunteer positioned on a couch 8 is introduced, the actual examination object O being located in the body of the patient or test volunteer. Even though the illustrated example depicts the torso of the examination object O, diffusion tensor imaging is often used to acquire brain images since it is particularly well-suited for imaging neurological structures.

The magnetic resonance scanner 2 is equipped conventionally with a basic field magnet system 4, a gradient system 6, an RF transmit antenna system 5, and an RF receive antenna system 7. In the exemplary embodiment shown, the RF transmit antenna system 5 is a whole-body coil permanently installed in the magnetic resonance scanner 2. In contrast, the RF receive antenna system 7 consists of local coils 7 that are to be arranged on the patient or test volunteer (symbolized here by just a single local coil 7 positioned on the body). The basic field magnet system 4 is embodied in this case in the conventional manner in such a way that it generates a basic magnetic field in the longitudinal direction of the patient, i.e., along the longitudinal axis of the magnetic resonance scanner 2 extending in the z-direction. The gradient system 6 comprises individually drivable gradient coils in the conventional manner to switch gradients in the x-, y-, or z-direction independently of one another.

The MRT system 1 shown here is a whole-body system comprising a patient tunnel into which a patient can be fully introduced. In principle, however, the aspects of the disclosure can also be used on other MRT systems, e.g., having a C-shaped housing with a side access opening. The only essential point is that corresponding images of the examination object O can be produced.

The magnetic resonance tomography system 1 also comprises a central control device 13, which controls the MR system 1. Said central control device 13 comprises a sequence control unit 14. This controls the succession of radiofrequency pulses (RF pulses) and gradient pulses as a function of a chosen pulse sequence or a succession of multiple pulse sequences for scanning a plurality of slices in a volume region of interest of the examination object within a measurement session. For example, such a pulse sequence can be specified and parameterized within a measurement or control protocol. Typically, various control protocols for different measurements or measurement sessions are stored in a memory 19 and can be selected by an operator (and possibly modified if necessary) and then used for performing the measurement. In the present case, the control device 13 contains pulse sequences to acquire raw data.

To output the individual RF pulses of a pulse sequence, the central control device 13 has a radiofrequency transmit device 15, which generates the RF pulses, amplifies them, and feeds them via a suitable interface (not shown in detail) into the RF transmit antenna system 5. To control the gradient coils of the gradient system 6 to switch the gradient pulses appropriately in accordance with the specified pulse sequence, the control device 13 has a gradient system interface 16. The sequence control unit 14 communicates in a suitable manner, e.g., by transmitting sequence control data SD, with the radiofrequency transmit device 15 and the gradient system interface 16 to execute the pulse sequence.

The control device 13 also comprises a radiofrequency receive device 17 (likewise communicating in a suitable manner with the sequence control unit 14) to receive magnetic resonance signals in a coordinated fashion using the RF receive antenna system 7 within the readout windows specified by the pulse sequence PS and in this way to acquire the raw data.

A reconstruction unit 18 accepts the acquired raw data in this case and reconstructs magnetic resonance image data therefrom. This reconstruction is generally performed based on parameters specified in the respective measurement or control protocol. For example, the image data can then be stored in a memory 19.

The details of how suitable raw data can be acquired by radiating RF pulses and switching gradient pulses and how MR images or parameter maps can be reconstructed therefrom are generally known to the person skilled in the art and are therefore not explained in greater depth here.

Operator control of the central control device 13 is possible by way of a terminal 11 comprising an input unit 10 and a display unit 9, via which terminal 11 the entire magnetic resonance tomography system 1 can therefore also be controlled by an operator. Magnetic resonance tomography images can also be displayed on the display unit 9, and measurements can be planned and started using the input unit 10, where necessary, in combination with the display unit 9.

The magnetic resonance tomography system 1, according to the aspects of the disclosure and, in particular, the control device 13 can furthermore also comprise a plurality of other components that are not shown here specifically but are typically present on systems of said type, such as a network interface, for example, to connect the system as a whole to a network and to enable raw data and/or image data or parameter maps, but also further data, such as, for example, patient-related data or control protocols, to be exchanged.

How suitable raw data can be acquired by radiating RF pulses and generating gradient fields and how magnetic resonance tomography images can be reconstructed therefrom is generally known to the person skilled in the art and is not explained in further detail here. Similarly, a vast array of measurement sequences, such as, e.g. EPI measurement sequences or other measurement sequences for generating diffusion-weighted images, are generally known to the person skilled in the art.

FIG. 2 shows a local coil 7 comprising two antennas A and a bite splint B, which is indicated between the two antennas A. A lower jaw is indicated as the examination object O. In an examination, the bite splint is placed into the mouth and retained by the teeth. The patient is then positioned into an MRT scanner 2 so that his or her head lies in the magnetic field and can be measured.

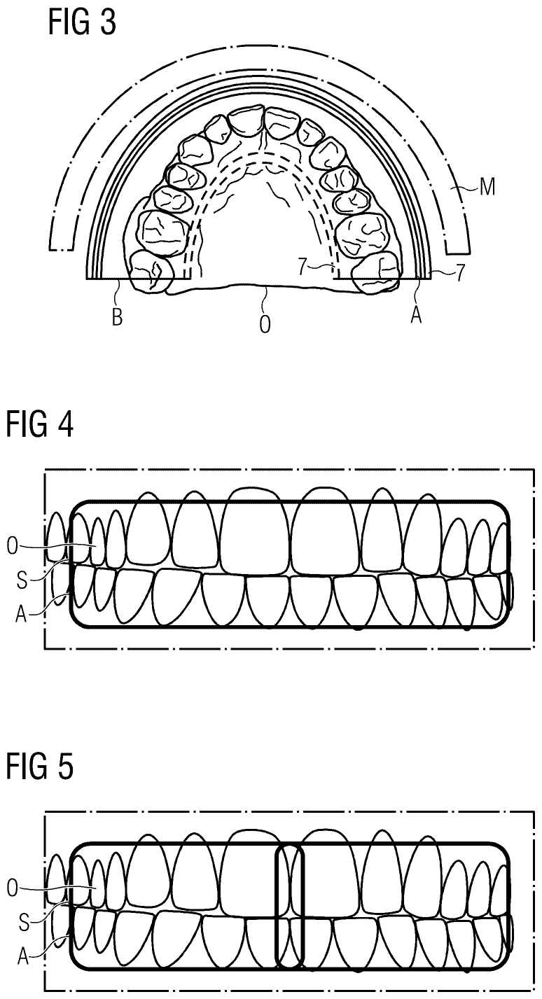

FIG. 3 shows an arrangement of the local coil 7 in an oral cavity from above. The coil could be a local coil 7, as shown in FIG. 2. The bite splint B protrudes into the region in which it can be retained by the teeth. Internally, indicated by a dashed line in the mouth's interior, an alternative local coil 7 would be situated in the oral cavity. Externally, the oral cavity environment M, in other words, e.g., the lips and cheeks, is indicated.

FIG. 4 shows a preferred antenna mold for a local coil 7 for use in the oral cavity. In this case, a single (curved) coil as coil structure S covers the teeth as antenna A.

FIG. 5 shows a preferred double antenna mold for a local coil 7 for use in the oral cavity. In this case, one coil of the coil structure S of the antenna A covers the upper jaw and another the lower jaw.

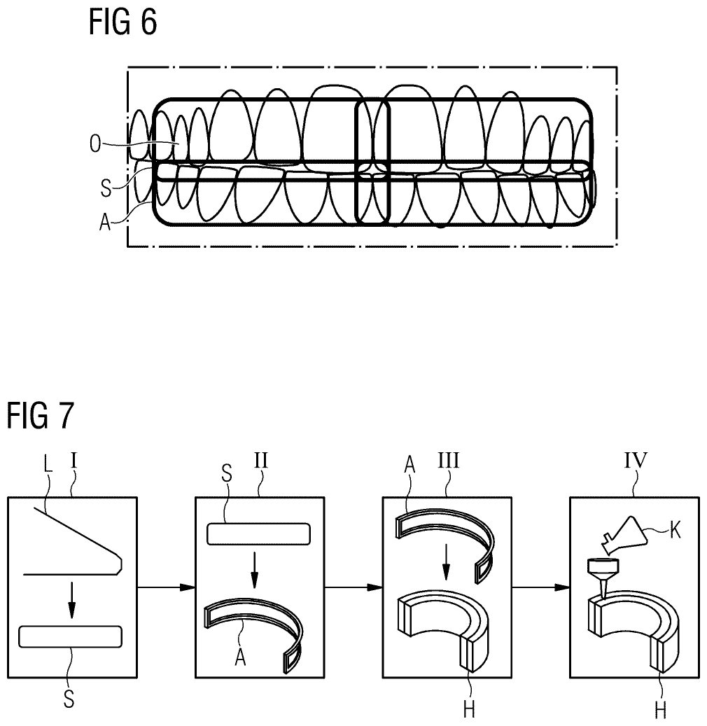

FIG. 6 shows a preferred quadruple antenna mold for a local coil 7 for use in the oral cavity. In this case, two coils of the coil structure S of the antenna A cover the upper jaw and two others the lower jaw.

FIG. 7 shows a block diagram of an exemplary embodiment of a method according to the aspects of the disclosure for producing a local coil 7 for an MRT measurement.

In step I, a coil structure S is shaped by repeatedly winding an elongate conductor L to form a coil. The resulting coil structure S is still flat in this case.

In step II, the flat coil structure S is shaped, in this case by bending, to form an antenna A for the MRT measurement. The antenna now looks as indicated, e.g., in FIGS. 2 and 3.

In step III, the curved antenna A is introduced into a cavity mold H. Said cavity mold H is designed to receive a liquid plastic material K, e.g., for the injection molding process. In this case, the antenna A is arranged in the cavity mold H so that it can be at least partially enveloped by liquid plastic material K.

In step IV, liquid plastic material K is poured in to fill the cavity mold H and cured, e.g., by cooling down the plastic material K. The antenna A is molded into the plastic material K, then results in the local coil 7.

In conclusion, it is pointed out once again that the methods described in detail in the foregoing, as well as the illustrated local coil 7, are simply exemplary embodiments which may be modified in the most diverse ways by the person skilled in the art without leaving the scope of the aspects of the disclosure. Furthermore, the use of the indefinite articles “a” or “an” does not exclude the possibility that the features in question may also be present more than once. Similarly, the terms “unit” and “module” do not rule out the possibility that the components in question consist of a plurality of cooperating subcomponents, which, if necessary, may also be distributed in space.

1. A method for producing a local coil for an MRT measurement, comprising:
shaping a coil structure from an elongate conductor;
shaping an antenna for the MRT measurement from the coil structure;
introducing the antenna into a cavity mold that is designed to receive a liquid plastic material, wherein the antenna is arranged in the cavity mold in such a way that it is at least partially encapsulatable by liquid plastic material; and
shaping the local coil by filling the cavity mold with a liquid plastic material and curing the plastic material.

2. The method of claim 1, wherein the shaping of the antenna comprises:
combining the antenna with a design mold, which is the cavity mold, by joining the antenna to the design mold.

3. The method of claim 2, wherein the shaping of the antenna comprises:
plastically deforming the design mold by compression molding with a heat application.

4. The method of claim 1, wherein the conductor is a wire, a flexible lead, or a conductor track on a substrate material and/or the coil structure is formed from a continuous conductor.

5. The method of claim 1, wherein the coil structure comprises a plurality of conductors that form a plurality of independent and electrically isolated antenna elements.

6. The method of claim 1, wherein the local coil is shaped using an injection molding process, a casting process, a foaming process, or a dip molding process.

7. The method of claim 1, wherein the antenna is shaped and introduced into the cavity mold such that:
endpieces of the conductor protrude from the cavity mold and are not encased by plastic material during the shaping of the local coil,
the antenna forms an inductance that is inductively connectable to an external inductance, and
the antenna forms an electrode that is usable according to its intended purpose for capacitive coupling to an external electrode.

8. The method of claim 7, wherein the plastic material or a substrate material of a conductor track of the conductor serves as a dielectric.

9. The method of claim 1, further comprising:
introducing an insertion element into the cavity mold before it is filled with the plastic material and/or the cavity mold is cast to create a cavity inside the local coil.

10. The method of claim 1, further comprising:
repeating a number of times the step of filling the mold with a liquid plastic material such that different plastic layers are superimposed on one another.

11. The method of claim 10, further comprising:
using a plurality of different plastic materials possessing different elastic properties in a cured state.

12. The method of claim 1, wherein a plurality of local coils of the same type are manufactured in different sizes and at the same time are produced in such a way that they have terminals at different geometric positions.

13. The method of claim 1, wherein the antenna is produced in such a way that it forms an additional inductance at a point that has little imaging impact.

14. The method of claim 13, wherein during manufacture of a plurality of local coils of the same type having different sizes, the antennas of the local coils are produced having different additional inductances such that the smallest antenna receives the greatest additional inductance.

15. The method of claim 1, wherein the local coil is produced in such a way that it comprises a shortening capacitor that is designed and connected to the antenna in such a way that the antenna has a predetermined impedance.

16. The method of claim 15, wherein the shortening capacitor is integrated into the antenna using a double-sided metallization or is produced using a discrete element.

17. A local coil shaped using the method of claim 1.

18. The local coil of claim 17, further comprising:
contacts for electrically contacting with an MRT interface and mechanically retaining structures which, in conjunction with the contacts, are designed to form a plug that serves to produce a mechanically reinforced plug-in connection with the interface.

19. The local coil of claim 17, wherein the local coil is embodied with regard to its antenna and the shape of the plastic material for insertion into a body orifice for insertion into an oral cavity or for rectal or vaginal insertion or insertion into a blood vessel.

20. The local coil of claim 17, further comprising:
mechanical or electrical structures at its surface formed to serve as triggers for a switch of an MRT interface or as electrical signal contacts.
